(19) United States
(12) Patent Application Publication
(10) Pub. No.: US 2026/0014175 A1
(43) Pub. Date: Jan. 15, 2026

(54) PHARMACEUTICAL COMPOSITION FOR ORAL ADMINISTRATION OF CANNABINOIDS

(71) Applicant: EMPOWERPHARM INC., Burlington (CA)

(72) Inventors: Chirag Rameshkumar SHAH, Burlington (CA); Hemang Rameshchandra JOSHI, Burlington (CA); Joseph SCHWARZ, North York (CA); Michael WEISSPAPIR, North York (CA)

(73) Assignee: EMPOWERPHARM INC., Burlington, ON (CA)

(21) Appl. No.: 18/875,377

(22) PCT Filed: Jun. 13, 2023
(86) PCT No.: PCT/CA2023/050819

§ 371 Date: Dec. 16, 2024

Related U.S. Application Data
(60) Provisional application No. 63/366,372, filed on Jun. 14, 2022.

(51) Int. Cl. A61K 31/00; A61K 9/20

(52) U.S. Cl. CPC A61K 31/658; A61K 9/2009; A61K 9/2013; A61K 9/2018; A61K 9/2054; A61K 9/2077; A61K 9/2095

(57) ABSTRACT

A pharmaceutical composition in the form of a compressed tablet for oral administration. The composition has an intragranular portion including a first porous solid carrier with a physiologically acceptable salt that is soluble in gastric fluid/an acidic aqueous media, and a self-nanoemulsifying drug delivery system (SNEDDS) adsorbed on the carrier. The SNEDDS includes: at least one cannabinoid; one or more solubilizing agents selected from physiologically acceptable organic compounds including: alcohols C8-C18, cyclic alcohols, aromatic alcohols, glycerides, polyethylene glycols, polyethylene glycol esters, aromatic esters, phenols, tocopherols, phospholipids, polyoxylglycerides, or polyoxyethylene stearates; one or more emulsifying agents selected from physiologically acceptable nonionic surfactants; and one or more carrier oils. The composition has an extragranular portion including: an optional second porous solid carrier including a physiologically acceptable salt that is soluble in gastric fluid/an acidic aqueous media; one or more disintegrants; one or more diluents; and one or more lubricants.

### CROSS-REFERENCE TO RELATED APPLICATIONS

The present patent application is a 371 National Stage with Application Number PCT/CA2023/050819 filed on Jun. 13, 2023, which claims priority to U.S. Provisional Application No. 63/366,372, filed Jun. 14, 2022, which is hereby incorporated by reference into the present disclosure.

### FIELD

The present invention pertains to a pharmaceutical composition comprising cannabinoids. More particularly, the present invention pertains to a pharmaceutical composition in the form of a compressed tablet for oral administration of cannabinoids, such as cannabidiol.

### BACKGROUND

There is increasing interest in the potential utility of cannabinoids to treat a variety of medical conditions. Cannabinoids can include endocannabinoids (which are endogenous cannabinoids naturally produced in the body by animals, such as humans), phytocannabinoids (such as those found in the Cannabis plant), and synthetic/semi-synthetic cannabinoids.

Cannabidiol (CBD) is a phytocannabinoid discovered in 1940. It is one of more than 110 identified cannabinoids in cannabis plants. CBD, 2-[(1R, 6R)-3-methyl-6-(1-methylethenyl)-2-cyclohexen-1-yl]-5-pentyl-1,3-benzenediol, is an isomer of (−)-trans-Δ9-tetrahydrocannabinol (THC; also known as delta-9-tetrahydrocannabinol or Δ9-THC); however, it does not produce the “high”, i.e. intoxicating effect. CBD has shown promise in the treatment of a number of diseases and disorders, such as anxiety, insomnia, and chronic pain. THC has also been used to treat a wide range of medical conditions.

While there are many dosage forms of cannabinoids, such as CBD and THC, oral formulations are preferred and may lead to better patient compliance than other dosage forms. However, oral formulations of cannabinoids, such as THC and CBD, can have poor solubility and poor bioavailability due to the lipophilic and highly hydrophobic nature of these and other cannabinoid compounds.

There is a need for alternative pharmaceutical compositions for oral administration of cannabinoids, such as CBD and THC.

This background information is provided for the purpose of making known information believed by the applicant to be of possible relevance to the present invention. No admission is necessarily intended, nor should be construed, that any of the preceding information constitutes prior art against the present invention.

### SUMMARY

In one aspect, there is provided a pharmaceutical composition in the form of a compressed tablet for oral administration having an intragranular portion and an extragranular portion, wherein:

the intragranular portion comprises: a first porous solid carrier comprising a physiologically acceptable salt that is soluble in gastric fluid or in an acidic aqueous media; a self-nanoemulsifying drug delivery system (SNEDDS) adsorbed on the carrier, wherein the SNEDDS comprises: at least one cannabinoid; one or more solubilizing agents selected from physiologically acceptable organic compounds comprising: alcohols C8-C18, cyclic alcohols, aromatic alcohols, glycerides, polyethylene glycols, polyethylene glycol esters, aromatic esters, phenols, tocopherols, phospholipids, polyoxylglycerides, or polyoxyethylene stearates; one or more emulsifying agents selected from physiologically acceptable nonionic surfactants; and one or more carrier oils; and

the extragranular portion comprises: an optional second porous solid carrier comprising a physiologically acceptable salt that is soluble in gastric fluid or in an acidic aqueous media; one or more disintegrants; one or more diluents; and one or more lubricants.

### BRIEF DESCRIPTION OF THE FIGURES

For a better understanding of the present invention including the progression of development to get to the end product, reference is made to the following description which is to be used in conjunction with the accompanying drawings, where:

FIG. 1 is a flowchart illustrating the process used for preparing the small-scale batches of compressed tablets for oral administration containing 10 mg, 25 mg, and 50 mg of cannabidiol (CBD).

FIG. 2 is a photo of exemplary 50 mg, 25 mg, and 10 mg compressed tablets for oral administration (in order from left to right in the photo) prepared according to Example 2.

FIG. 3a is a chart illustrating dissolution behaviour of 10 mg CBD tablets following 2 months (2M) of storage under room temperature (“RT”; 25° C./60% RH) or accelerated storage conditions (“Acc”; 40° C. 75% RH).

FIG. 3b is a chart illustrating dissolution behaviour of 25 mg CBD tablets following 0 months (OM) and 2 months (2M) of storage under room temperature (“RT”; 25° C./60% RH) or accelerated storage conditions (“Acc”; 40° C. 75% RH).

FIG. 4 is a photo of exemplary 25 mg and 10 mg compressed tablets for oral administration (in order from left to right in the photo) prepared according to Example 3.

FIGS. 5a through 5d are flowcharts illustrating another embodiment of a process used for preparing compressed tablets for oral administration containing 10 mg and 25 mg of cannabidiol (CBD).

FIG. 6a is a chart illustrating dissolution behaviour of 10 mg CBD tablets prepared according to Example 3 (“Original Formula”) and Example 4 (“Optimized Formula”).

FIG. 6b is a chart illustrating dissolution behaviour of 25 mg CBD tablets prepared according to Example 3 (“Original Formula”) and Example 4 (“Optimized Formula”).

FIG. 6c is a chart illustrating dissolution behaviour of 25 mg CBD tablets prepared according to Example 3 (“Original Formula”) and Example 4 (“Optimized Formula”) versus INSTACANN®25 mg CBD tablets manufactured by CannTab Therapeutics Limited.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) ingredient(s) and/or elements(s) as appropriate.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

The term “cannabinoid” refers to a chemical compound that shows direct or indirect activity at a cannabinoid receptor. Cannabinoids can include endocannabinoids (which are endogenous cannabinoids naturally produced in the body by animals, such as humans), phytocannabinoids (such as those found in the Cannabis plant), and synthetic/semi-synthetic cannabinoids.

The term “eutectic” comes from the Greek words for “easily melted” (Atkins, Peter W. (1990). Physical Chemistry. 4^{th} ed.: Oxford University Press). A “eutectic mixture” is conventionally defined as the one mixture of a set of substances able to dissolve in one another as liquids that, of all such mixtures, liquefies at the lowest temperature (https://www.britannica.com/science/eutectic; accessed Jun. 5, 2022). Thus, an eutectic mixture can be defined as a mixture of two or more substances that melts at a temperature lower than the melting point of any single component or any other mixture of them.

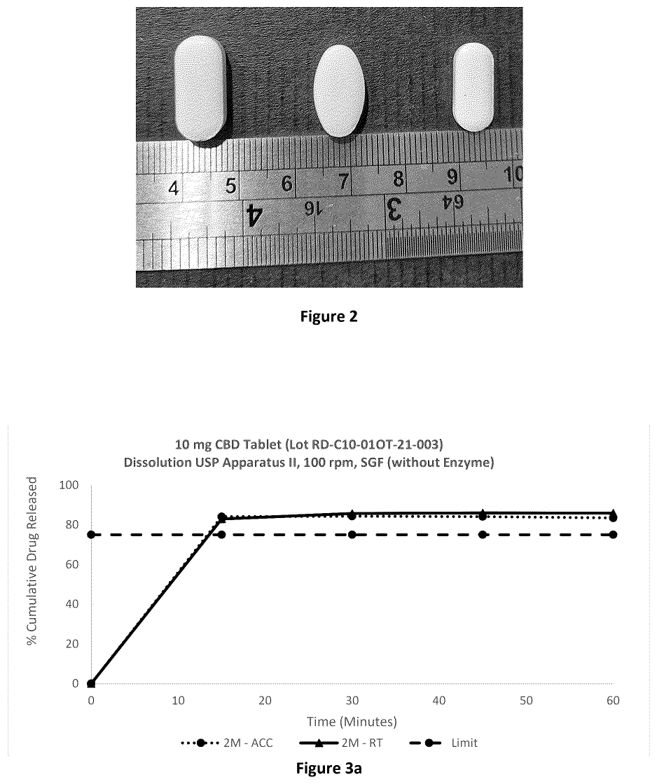

In some instances, the eutectic melting point may lie within a range of compositions—see, for example, WO2003/041632 to Texas Tech University (FIG. 2, [0015] and [0036]).

In one embodiment, there is provided a pharmaceutical composition in the form of a compressed tablet for oral administration having an intragranular portion and an extragranular portion, wherein:
the intragranular portion comprises: a first porous solid carrier comprising a physiologically acceptable salt that is soluble in gastric fluid or in an acidic aqueous media; a self-nanoemulsifying drug delivery system (SNEDDS) adsorbed on the carrier, wherein the SNEDDS comprises: at least one cannabinoid, such as cannabidiol (CBD); one or more solubilizing agents selected from physiologically acceptable organic compounds comprising: alcohols C8-C18, cyclic alcohols, aromatic alcohols, glycerides, polyethylene glycols, polyethylene glycol esters, aromatic esters, phenols, tocopherols, phospholipids, polyoxylglycerides, or polyoxyethylene stearates; one or more emulsifying agents selected from physiologically acceptable nonionic surfactants; and one or more carrier oils; and
the extragranular portion comprises: an optional second porous solid carrier comprising a physiologically acceptable salt that is soluble in gastric fluid or in an acidic aqueous media; one or more disintegrants; one or more diluents; and one or more lubricants.

In one embodiment, the at least one cannabinoid is selected from cannabidiol (CBD), tetrahydrocannabinol (THC; including the two isoforms Δ9-THC, Δ8-THC, preferably Δ9-THC), cannabigerol (CBG), cannabidiolic acid (CBDA), tetrahydrocannabinolic acid (THCA), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives/analogs thereof, or mixtures thereof. In another embodiment, the at least one cannabinoid is selected from CBD, Δ9-THC, CBG, or mixtures thereof. In another embodiment, the at least one cannabinoid is CBD.

In one embodiment, the CBD is a plant based isolate. In other embodiments, the CBD is synthetic/semi-synthetic CBD or bio-synthetic CBD.

In one embodiment, the first porous solid carrier and/or optional second porous solid carrier comprises porous dibasic calcium phosphate or porous tribasic calcium phosphate. In another embodiment, the first porous solid carrier and the optional second porous solid carrier comprise porous dibasic calcium phosphate, wherein the dibasic calcium phosphate is anhydrous. In one embodiment, the second porous solid carrier is present and can adsorb any liquid phase squeezed from the intragranular portion during tablet compression. In one embodiment, the second porous solid carrier is absent. In another embodiment, the first porous solid carrier and/or optional second porous solid carrier are Fujicalin® (calcium phosphate, dibasic anhydrous). Fujicalin® is a particularly advantageous adsorbent, as it has a capacity to adsorb 1 part of oil in 3 parts of Fujicalin®.

In one embodiment, the one or more solubilizing agents can be selected from alcohols C8-C18 such as cetyl C16, behenyl C22, C8, C10, or C12 alcohols; cyclic alcohols such as L-menthol (1R,2S,5R)-2-isopropyl-5-methylcyclohexanol), or borneol (bicyclic alcohol); aromatic alcohols such as benzyl alcohol; glycerides such as glyceryl monostearate (GMS), glyceryl monolinoleate (GML), glyceryl monooleate (GMO), glyceryl monocaprylate (GMC), cocoa butter, or mono- and diglycerides C8-C18; polyethylene glycols such as PEG 3350; polyethylene glycol esters such as PEG-32 stearate, PEG-40 stearate, or Vitamin E succinate PEG-1000 ester (TPGS); aromatic esters such as anisole or anethole; phenols such as butylated hydroxytoluene, butylated hydroxyanisole, or thymol; tocopherols such as D-alpha Tocopherol or D-alpha tocopheryl acetate; phospholipids such as lecithin, phosphatidyl choline, or phosphatidyl ethanolamine; polyoxylglycerides such as lauroyl polyoxylglycerides or caprylocaproyl polyoxylglycerides; or polyoxyethylene stearates.

In another embodiment, the one or more solubilizing agents are selected from polyoxylglycerides, such as lauroyl polyoxylglycerides and caprylocaproyl polyoxylglycerides, including lauroyl polyoxyl-32 glycerides (e.g. Gelucire 44/14), and caprylocaproyl polyoxyl-8 glycerides (e.g. Acconon MC8-2 from Abitec; Labrasol from Gattefosse). In another embodiment, the one or more solubilizing agents are selected from polyoxyethylene stearates, such as polyoxyl-32 stearate (e.g. Gelucire 48/16). In another embodiment, the one or more solubilizing agents comprise glyceryl monolinoleate (e.g. Maisine CC). In another embodiment, the one or more solubilizing agents can be a combination of any of the foregoing. In still another embodiment, the one or more solubilizing agents are selected from lauroyl polyoxyl-32 glycerides (e.g. Gelucire 44/14).

In one embodiment, the one or more emulsifying agents are selected from physiologically acceptable nonionic surfactants, such as lecithin (e.g. soy lecithin, such as Phospholipon 90G), polyoxyl 40 hydrogenated castor oil (e.g. Kolliphor RH 40), polysorbate 60 (e.g. Kolliphor PS60, Tween-60), sorbitan monooleate (e.g. Montane 80 PHA Premium), polysorbate 20, polysorbate 80, sorbitan monostearate, or combinations thereof. In another embodiment, the one or more emulsifying agents are selected from Phospholipon 90G, Kolliphor RH 40, Kolliphor PS60/Tween 60, or Montane 80 PHA Premium, or combinations thereof.

In one embodiment, the one or more carrier oils are selected from medium chain triglycerides (MCT; such as Kollisolv MCT 70, or Labrafac Lipophile WL 1349), almond oil, peppermint oil, fractionated coconut oil, sesame oil, pumpkin seed oil, avocado oil, olive oil, corn oil, soybean oil, cottonseed oil, safflower oil, L-menthol-containing oil, spearmint oil, or combinations thereof. In another embodiment, the one or more carrier oils are selected from Kollisolv MCT 70, almond oil, peppermint oil, or combinations thereof.

In one embodiment, the intragranular portion comprises one or more antioxidants. In another embodiment, the one or more antioxidants are selected from butylated hydroxytoluene, butylated hydroxyanisole, ascorbic acid, ascorbyl palmitate, tocopherols and tocopherol esters, propyl gallate, or tertiary butyl hydroquinone.

In one embodiment, the intragranular portion further comprises one or more of an intragranular diluent, and a chelating agent. In another embodiment, the intragranular portion comprises an intragranular diluent selected from microcrystalline cellulose (MCC), polyols, polysaccharides, starches, sugars, or amino acids, or combinations thereof. In another embodiment, the intragranular diluent is selected from glycine, microcrystalline cellulose, or combinations thereof. In still another embodiment, the intragranular portion comprises a chelating agent selected from ethylenediaminetetraacetic acid (EDTA) and EDTA salts, ethylene glycol-bis(O-aminoethyl ether)-N,N,N′,N′-tetraacetic acid (EGTA) and EGTA salts, citric acid and citrates, or combinations thereof. In another embodiment, the intragranular portion comprises an intragranular disintegrant selected from croscarmellose sodium or sodium starch glycolate.

In still another embodiment, the intragranular portion comprises a polymeric binder, applied on an intragranular admixture comprising the first porous solid carrier having the SNEDDS adsorbed thereto; the binder comprising a film-forming agent selected from low viscosity hydroxylpropyl cellulose, or hydroxyethyl cellulose; optionally, wherein the intragranular admixture comprises a further polymeric binder in admixture with the first porous solid carrier, wherein the further polymeric binder is selected from selected from low viscosity hydroxylpropyl cellulose, or hydroxyethyl cellulose.

In another embodiment, the intragranular portion optionally comprises one or more intragranular disintegrants, and the one or more intragranular disintegrants and the one or more disintegrants in the extragranular portion are independently selected from croscarmellose sodium or sodium starch glycolate.

In yet another embodiment, the one or more diluents in the extragranular portion are selected from microcrystalline cellulose (MCC), polyols, polysaccharides, starches, sugars, or amino acids.

In still yet another embodiment, the one or more lubricants are selected from sodium lauryl sulfate, polyethylene glycol (PEG) 3350, PEG 6000, PEG 8000, or sodium stearyl fumarate.

In another embodiment, the pharmaceutical composition is essentially free from silicon dioxides, silicates, polyvinylpyrrolidone (PVP) polymers, or copolymers of polyvinylpyrrolidone—for example, the pharmaceutical composition contains less than about 0.5% w/w silicon dioxides, silicates, polyvinylpyrrolidone (PVP) polymers, or copolymers of polyvinylpyrrolidone, or less than about 0.25% w/w, silicon dioxides, silicates, polyvinylpyrrolidone (PVP) polymers, or copolymers of polyvinylpyrrolidone.

In yet another embodiment: the at least one cannabinoid, such as cannabidiol (CBD), is present in an amount of from about 1% w/w to about 10% w/w of the tablet, or of from about 2% w/w to about 4% w/w of the tablet; the first porous solid carrier and the optional second porous solid carrier are collectively present in an amount of from about 30% w/w to about 75% w/w of the tablet, or of from about 35% w/w to about 75% w/w of the tablet, or of from about 40% w/w to about 75% w/w of the tablet, or of from about 35% w/w to about 45% w/w of the tablet; the one or more solubilizing agents are collectively present in an amount of from about 0.1% w/w to about 5% w/w of the tablet, or of from about 0.5% w/w to about 5% w/w of the tablet, or of from about 0.1% w/w to about 0.3% w/w of the tablet; the one or more emulsifying agents are collectively present in an amount of from about 2% w/w to about 15% w/w of the tablet, or of from about 4% w/w to about 6% w/w of the tablet; the one or more carrier oils are collectively present in an amount of from about 0.1% w/w to about 10% w/w of the tablet, or of from about 2% w/w to about 10% w/w of the tablet, or of from about 0.2% w/w to about 0.4% w/w of the tablet; the intragranular portion comprises one or more intragranular disintegrants, and the one or more intragranular disintegrants and the one or more disintegrants in the extragranular portion are collectively present in an amount of from about 1% w/w to about 10% w/w of the tablet, or of from about 7% w/w to about 9% w/w of the tablet; the intragranular portion comprises one or more intragranular diluents, and the one or more intragranular diluents and the one or more diluents in the extragranular portion are collectively present in an amount of from about 5% w/w to about 40% w/w of the tablet, or of from about 25% w/w to about 35% w/w of the tablet; and the one or more lubricants are collectively present in an amount of from about 1% w/w to about 10% w/w of the tablet, or of from about 1% w/w to about 5% w/w of the tablet, or of from about 1% w/w to about 7% w/w of the tablet, or of from about 5% w/w to about 7% w/w of the tablet. In another embodiment, the pharmaceutical composition further comprises the polymeric binder and, optionally, the further polymeric binder as defined above, wherein the polymeric binder and the further polymeric binder are collectively present in an amount of from about 0.5% w/w to about 10% w/w of the tablet, or of from about 5% w/w to about 10% w/w of the tablet; or of from about 7% w/w to about 9% w/w of the tablet.

In still yet another embodiment, a dissolution rate of the at least one cannabinoid (e.g. CBD) in the compressed tablet for oral administration is such that, when tested using USP Apparatus II (paddles) set to a rotation speed of 100 rpm in 900 mL of simulated gastric fluid with no enzyme at 370° C., at least 70% of the at least one cannabinoid dissolves in 30 minutes or less, or at least 75% of the at least one cannabinoid dissolves in 30 minutes or less. In another embodiment, at least 70% of the at least one cannabinoid (e.g. CBD) dissolves in 15 minutes or less, or at least 75% of the at least one cannabinoid dissolves in 15 minutes or less.

In one embodiment, the compressed tablet releases an oil-in-water nanoemulsion containing the at least one cannabinoid (e.g. CBD) upon contact with gastric fluid or acidic water media, wherein the at least one cannabinoid remains dissolved in the discontinuous phase of the formed oil-in-water nanoemulsion.

In another embodiment, the at least one cannabinoid (e.g. CBD) is present in the compressed tablet an amount of from about 5 mg to about 100 mg.

In another embodiment, there is provided a process for preparing the pharmaceutical compositions disclosed herein, the process comprising: combining the at least one cannabinoid (e.g. CBD) and the one or more solubilizing agents under heating conditions to at least partially solubilize the at least one cannabinoid, forming a first mixture; combining the one or more emulsifying agents, and one or more carrier oils with the first mixture under heating conditions, to form the SNEDDS; combining the SNEDDS with the first porous solid carrier to adsorb the SNEDDS to the first porous solid carrier; combining the first porous solid carrier having the SNEDDS adsorbed thereon with the second porous solid carrier (optional), the one or more disintegrants, the one or more diluents, and the one or more lubricants, to form a pre-tabletting mixture; and forming the compressed tablet from the pre-tabletting mixture.

In another embodiment, a process for preparing the pharmaceutical compositions described herein, the process comprising: combining the at least one cannabinoid (e.g. CBD), the one or more solubilizing agents, and the one or more carrier oils under heating conditions to at least partially solubilize the at least one cannabinoid, forming a first mixture; combining the one or more emulsifying agents with the first mixture under heating conditions, to form the SNEDDS; combining the SNEDDS with the first porous solid carrier to adsorb the SNEDDS to the first porous solid carrier; combining the first porous solid carrier having the SNEDDS adsorbed thereon with the second porous solid carrier, if present, the one or more disintegrants, the one or more diluents, and the one or more lubricants, to form a pre-tabletting mixture; and forming the compressed tablet from the pre-tabletting mixture. In another embodiment of this process, the second porous solid carrier is absent.

CBD has good solubility (up to 40%) in different oils. However, CBD solution in oil has a tendency to oversaturation; CBD may crystallize and precipitate from the oversaturated solutions within weeks/months. CBD solubility in oils may also decrease in presence of some polar additives which are used in pharmaceutical compositions, e.g., surfactants.

It is believed that CBD may form liquid or low melting eutectic mixtures with some pharmaceutically acceptable compounds, such as solubilizing agents selected from physiologically acceptable organic compounds comprising: alcohols C₈-C₁₈, cyclic alcohols, aromatic alcohols, glycerides, polyethylene glycols, polyethylene glycol esters, aromatic esters, phenols, tocopherols, phospholipids, polyoxylglycerides, or polyoxyethylene stearates. Eutectic mixtures with CBD are expected to have better solubility in oils in the presence of surfactants than CBD itself. As such, it is believed that combining CBD with the above-noted solubilizing agents in certain amounts in the presence of heat results in a composition that melts at a temperature lower than the melting point of either CBD or the solubilizing agents and/or results in a composition that allows the CBD to remain solubilized in an oil phase for extended period of time.

Gelucire 44/14 (Lauroyl polyoxyl-32 glyceride) is a solubilizing agent (and potential eutectic mixture (EM)-forming excipient) that can be used to form CBD-loaded SNEDDS for incorporation into compressed tablets for oral administration. Gelucire 44/14 has a melting point of about 38-44° C., and CBD has a melting point of about 66-68° C. The combined mixture of Gelucire 44/14 and CBD was determined to have a melting point of about 18-22° C. at a weight ratio 2:1 (CBD:Gelucire 44/14). A weight ratio of 1:1 (CBD:Gelucire 44/14) was also found to remain in liquid form when the combination of CBD and Gelucire 44/14 was heated and then cooled to room temperature.

CBD is completely dissolved in the SNEDDS composition (oil phase composition) disclosed herein, and remains in the dissolved state in the SNEDDS composition. The CBD SNEDDS formulation is a viscous liquid or semi-solid material at room temperature, and is in a liquid form at 37° C. Thus, the compressed tablet for oral administration disclosed herein can be viewed as a “liquisolid tablet”. The tablets disclosed herein exhibit excellent dissolution behaviour, and are expected to be high quality treatment options within the medical cannabis market.

### EXAMPLES

### Materials & Methods

Pharmaceutical-grade CBD (plant based isolate) was obtained from a Canadian supplier and used in examples below. Table 1 below provides a listing of other ingredients used in the compositions described herein, including the brand name/grade under which they were purchased.

TABLE 1
listing of ingredients used in the compositions

| Ingredient | Brand Name/Grade |
|---|---|
| MCT oil | Kollisolv MCT 70/USP EP; |
| | Labrafac Lipophile WL 1349 |
| | used in Example 4 |
| Polysorbate 60 | Kolliphor PS 60/USP EP JP |
| Polyoxy1 40 | Kolliphor RH 40/USP EP |
| Hydrogenated | |
| castor oil | |
| Sodium Lauryl | Kolliphor SLS/USP EP JP |
| sulfate | |
| Sorbitan monooleate | Span 80 NF |
| D-alpha Tocopherol | Covitol 1360 USP, FCC |
| acetate | Novatol 1360 USP, FCC |
| D-alpha Tocopherol | Covitol F 1490 USP, FCC |
| | Novatol 1490 USP, FCC |
| Lauroyl polyoxyl-32 | Gelucire 44/14 USP/NF, EP |
| glycerides | |
| Ascorbyl palmitate | Ascorbic acid 6-palmitate NF |
| NF | |
| Butylated | BHT, FCC grade |
| hydroxytoluene | |
| Porous dibasic | Fujicalin ® USP, EP, JP |
| calcium phosphate | |
| Microcrystalline | Avicel ® PH-102 USP/NF; |
| cellulose | Vivapur 102 used for Example 4 |
| Crosscarmelose | Ac-Di-Sol ® USP/NF; Vivasol |
| sodium | GF (used in Example 4) |
| Glycine | Glycine USP |
| Peppermint oil | Peppermint oil FCC |
| Almond oil | Almond oil from Prunus dulcis, |
| | USP, FCC |
| Lecithin | Phospholipon 90G |
| Ethyl alcohol | Ethanol anhydrous USP/NF |
| anhydrous | |
| Hydroxypropyl | Nisso HPC SSL-SF |
| cellulose low | |
| viscosity | |
| EDTA sodium | EDTA disodium USP/NF |
| Sodium Stearyl | Lubripharm SSF |
| Fumarate, USP-NF | |

TABLE 2
Dissolution media

| Medium | Composition |
|---|---|
| Simulated gastric fluid | sodium chloride (2 g/L) and hydrochloric acid |
| (SGF) with no enzymes | to pH 1.2 |
| Simulated intestinal | KH₂PO4 (68.05 g), NaOH (8.96 g), deionized |
| fluid (SIF) | water to 10.0 L, pH 6.81 |
| Fasted state simulated | sodium taurocholate (3 mM), lecithin (0.75 |
| intestinal fluid (FaSSIF) | mM), NaOH (pellets; 0.174 g), NaH₂PO₄•H₂O |
| | (1.977 g), NaCl (3.093 g), purified water |
| | (qs. 500 mL), pH of about 6.50 |

### Example 1—Determining the Compatibility of CBD and/or CBD-Containing Self-Nanoemulsifying Drug Delivery Systems (SNEDDS) with Various Excipients

A number of different compositions were prepared and tested in order to determine the compatibility of CBD and/or CBD-containing self-nanoemulsifying drug delivery systems (SNEDDS) with various excipients. It was surprisingly found that a number of excipients are incompatible with CBD and/or CBD-containing SNEDDS.

### 1) Silicon Dioxides and Magnesium Aluminum Silicates

Based on experiments carried out it was clearly confirmed that all tested silicon dioxides (Cab-O-Sil, Syloid 350 XP, Syloid 244) and mesoporous MgAl silicate (Neusilin) are incompatible with CBD/CBD-containing SNEDDS. CBD was not released completely from binary mixtures of these excipients in acidic (simulated gastric fluid, SGF), neutral (simulated intestinal fluid, SIF), or fasting simulated intestinal fluid (FaSSIF), even in the presence of surfactant.

### 2) Polyvinyl Pyrrolidone (PVP) and its Copolymers

PVP and copolymer PVP-Vinylacetate (Copovidone, PVP-VA 64) were also found to be incompatible with CBD SNEDDS, resulting in the formation of a coarse emulsion upon dissolution. As the skilled worker will appreciate, fine emulsions are absorbed much better than coarse emulsions. It was determined that these components interact with CBD and can form insoluble complexes, preventing adequate dissolution of the dosage forms, especially in neutral conditions. Similar behavior can be expected for cross-linked PVP (Crosspovidone), one of the most effective disintegrants.

### 3) Microcrystalline Cellulose (MCC)

While CBD-containing SNEDDS can be adsorbed on microcrystalline cellulose (MCC), it was found that the active compound cannot be completely released, especially in a neutral environment (SIF). Up to 60% of CBD remained associated with MCC at least for 1 hour after beginning of a dissolution. Nevertheless, due to the positive impact of MCC on tablet compressibility and hardness, it was determined that MCC can be used in CBD SNEDDS tablets together with Fujicalin® (referenced below), with the amount of Fujicalin® preferably being in excess of the amount of MCC.

### 4) Porous Dibasic Calcium Phosphate (Fujicalin®)

Fujicalin® demonstrated excellent adsorption properties for CBD SNEDDS composition (also demonstrated in Examples below). CBD was completely released from Fujicalin® based formulations in SGF and up to 60-80% in SIF and FaSSIF.

### 5) Other Excipients:

Crosslinked carboxymethyl cellulose (Crosscarmellose, Ac-Di-Sol), Hydroxypropyl cellulose (HPC), Sodium lauryl sulfate (SLS), Glycine, EDTA sodium were also tested, and all of these excipients showed no signs of incompatibility with CBD/CBD-SNEDDS formulations.

Conclusions: In view of the foregoing, it is preferred that the present pharmaceutical compositions should be essentially free from silicon dioxides, silicates, polyvinylpyrrolidone (PVP) polymers, or copolymers of polyvinylpyrrolidone.

### Example 2—Preparation of Small-Scale Batches of Compressed Tablets for Oral Administration Containing 10 mg, 25 mg, and 50 mg of Cannabidiol (CBD)

As noted above, CBD has good solubility (10-40%) in different oils. However, CBD solution in oil has tendency to oversaturation; CBD may crystallize and precipitate from the oversaturated solutions within the time (weeks or even months). CBD solubility in oils may also decrease in presence of some polar additives which are used in pharmaceutical compositions, e.g., surfactants.

As noted above, it is believed that CBD may form liquid or low melting eutectic mixtures with some pharmaceutically acceptable compounds, such as solubilizing agents selected from physiologically acceptable organic compounds comprising: alcohols C₈-C₁₈, cyclic alcohols, aromatic alcohols, glycerides, polyethylene glycols, polyethylene glycol esters, aromatic esters, phenols, tocopherols, phospholipids, polyoxylglycerides, or polyoxyethylene stearates. Eutectic mixtures with CBD are expected to have better solubility in oils in the presence of surfactants than CBD itself. As such, it is believed that combining CBD with the above-noted solubilizing agents in certain amounts in the presence of heat results in a composition that melts at a temperature lower than the melting point of either CBD or the solubilizing agents and/or results in a composition that allows the CBD to remain solubilized in an oil phase for extended period of time.

Gelucire 44/14 (Lauroyl polyoxyl-32 glyceride) was selected as a solubilizing agent (and potential eutectic mixture (EM)-forming excipient) for development of CBD-loaded SNEDDS for incorporation into compressed tablets for oral administration. The mixture was prepared by combining Gelucire 44/14 and crystalline CBD, with small amounts of antioxidants, and heating the mixture at 50-55° C. until the clear liquid was formed. Gelucire 44/14 has a melting point of about 38-44° C., and CBD has a melting point of about 66-68° C. The combined mixture of Gelucire 44/14 and CBD was determined to have a melting point of about 18-22° C. at a weight ratio 2:1 (CBD:Gelucire 44/14). The mixture contained 66% of CBD by weight and remained liquid at room temperature for a long time (at least one year).

Formulations of CBD loaded self-nanoemulsifying compositions, incorporated into compressed tablets and containing 10, 25 and 50 mg CBD were developed on a laboratory scale, having a batch size of approximately 3 kg. Tables 2-4 below outline the components present in the 10 mg, 25 mg, and 50 mg tablets.

First, a CBD SNEDDS concentrate was prepared. In order to reach good self-emulsifying properties, the above-noted mixture of CBD-Gelucire 44/14 was combined with surfactants, oils and lecithin to form an oil phase (CBD SNEDDS). The prepared CBD SNEDDS contained about 20% of CBD and remained liquid at room temperature.

TABLE 3
Self-nanoemulsifying composition
contains about 1 mg CBD per 4.67 mg

| | |
|---|---|
| Cannabidiol (CBD) | 21.59% |
| Lauroyl polyoxyl-32 glycerides (Gelucire 44/14) | 10.65% |
| MCT oil | 16.01% |
| Tocopheryl Acetate, d-alpha | 1.86% |
| Tocopherol, d-alpha | 0.74% |
| Lecithin | 1.12% |
| Polyethoxylated hydrogenated castor oil (Kolliphor RH-40) | 8.93% |
| Butylated hydroxytoluene | 0.11% |
| Ascorbyl palmitate | 0.13% |
| Peppermint oil FCC | 3.42% |
| Almond oil | 6.40% |
| PEG-20 sorbitan monostearate (Polysorbate 60) | 17.87% |
| Sorbitan monooleate (Span 80) | 11.17% |
| Total | 100.00% |

Numerous different SNEDDS with CBD-Gelucire 44/14 were prepared. A preferred composition as outlined in Tables 2-4 below contained polysorbate 60 (Tween-60), polyethoxylated hydrogenated castor oil (Kolliphor RH-40), sorbitan monooleate (Span-80) and soy lecithin (Phospholipon 90G) as surfactants and medium chain triglycerides (MCT oil), almond oil and peppermint oil as oily components. Similar SNEDDS compositions without lecithin were found to have good self-nanoemulsifying properties in most water media, such as SGF, SIF, and FaSSIF (see Materials & Methods), as well as saliva, but with increasing ionic strength formed only coarse emulsions. The presence of lecithin showed low influence on retaining of CBD on the absorbents—i.e. compositions including lecithin exhibited good dissolution behaviour.

In view of the studies conducted in Example 1, Fujicalin® (porous anhydrous dibasic calcium phosphate) was selected as an efficient absorbent for incorporation of CBD SNEDDS into granulation for the preparation of compressed tablets for oral administration.

TABLE 4
Composition of 50 mg CBD SNEDDS TABLET
PER STAGE

| 50 mg CBD tablet | mg/tab |
|---|---|
| OIL PHASE | |
| CBD | 50 |
| Gelucire 44/14 | 25 |
| MCT | 36 |
| D-alpha-Tocopheryl acetate | 4.5 |
| D-alpha Tocopherol | 1.8 |
| Soy Lecithin 90G | 2.7 |
| MCT for Lecithin dissolving | 2.7 |
| Kolliphor RH-40 | 21.5 |
| Polysorbate 60 | 43 |
| Span 80 | 26 |
| Peppermint oil | 12 |
| Almond oil | 15 |
| BHT | 0.25 |
| Ascorbyl palmitate | 0.25 |
| GRANULATION | |
| Fujicalin ® | 600 |
| Ac-Di-Sol | 10 |
| EDTA | 5 |
| HPC SSL SF | 20 |
| EtOH | 80 |
| EXTRAGRANULAR | |
| Fujicalin ® | 150 |
| MCC Avicel PH-102 | 40 |
| Ac-Di-Sol | 40 |
| Glycine | 25 |
| Sodium lauryl sulfate (SLS) | 15 |
| Total | 1145.7 |

CUMULATIVE

| 50 mg CBD tablet | mg/tab | % |
|---|---|---|
| OIL PHASE | | |
| CBD | 50 | 4.36% |
| Gelucire 44/14 | 25 | 2.18% |
| MCT | 36 | 3.14% |
| D-alpha-Tocopheryl acetate | 4.5 | 0.39% |
| D-alpha Tocopherol | 1.8 | 0.16% |
| Soy Lecithin 90G | 2.7 | 0.24% |
| MCT for Lecithin dissolving | 2.7 | 0.24% |
| Kolliphor RH-40 | 21.5 | 1.88% |
| Polysorbate 60 | 43 | 3.75% |
| Span 80 | 26 | 2.27% |
| Peppermint oil | 12 | 1.05% |
| Almond oil | 15 | 1.31% |
| BHT | 0.25 | 0.022% |
| Ascorbyl palmitate | 0.25 | 0.022% |
| SOLID PHASE COMPONENTS | | |
| Fujicalin ® | 750 | 65.46% |
| Ac-Di-Sol | 50 | 4.36% |
| EDTA | 5 | 0.44% |
| HPC SSL SF | 20 | 1.75% |
| EtOH | 80 | N/A |
| MCC Avicel PH-102 | 40 | 3.49% |
| Glycine | 25 | 2.18% |
| Sodium lauryl sulfate (SLS) | 15 | 1.31% |
| Tablet weight | 1145.7 | 100.00% |

TABLE 5
Composition of 25 mg CBD SNEDDS TABLET
PER STAGE

| 25 mg tablet | mg/tab |
|---|---|
| OIL PHASE | |
| CBD | 25 |
| Gelucire 44/14 | 12.5 |
| MCT | 18 |
| D-alpha-Tocopheryl acetate | 2.25 |
| D-alpha Tocopherol | 0.9 |
| Soy Lecithin 90G | 1.35 |
| MCT for Lecithin | 1.35 |
| Kolliphor RH-40 | 10.75 |
| Polysorbate 60 | 21.5 |
| Span 80 | 13 |
| Peppermint oil | 6 |
| Almond oil | 7.5 |
| BHT | 0.125 |
| Ascorbyl palmitate | 0.125 |
| GRANULATION | |
| Fujicalin ® | 350 |
| MCC Avicel PH-102 | 25 |
| Glycine USP | 10 |
| Ac-Di-Sol | |
| EDTA | 2.5 |
| 20% HPC SSL-SF in 95% EtOH | 10 |
| EtOH | 40 |
| EXTRAGRANULAR | |
| Fujicalin ® | 100 |
| MCC Avicel PH-102 | 100 |
| Ac-Di-Sol | 20 |
| Glycine USP | |
| Sodium lauryl sulfate (SLS) | 7.5 |
| Total | 745.35 |

CUMULATIVE

| 25 mg tablet | mg/tab | % |
|---|---|---|
| OIL PHASE | | |
| CBD | 25 | 3.35% |
| Gelucire 44/14 | 12.5 | 1.68% |
| MCT | 18 | 2.41% |
| D-alpha-Tocopheryl acetate | 2.25 | 0.30% |
| D-alpha Tocopherol | 0.9 | 0.12% |
| Soy Lecithin 90G | 1.35 | 0.18% |
| MCT for Lecithin | 1.35 | 0.18% |
| Kolliphor RH-40 | 10.75 | 1.44% |
| Polysorbate 60 | 21.5 | 2.88% |
| Span 80 | 13 | 1.74% |
| Peppermint oil FCC grade | 6 | 0.80% |
| Almond oil | 7.5 | 1.01% |
| BHT | 0.125 | 0.017% |
| Ascorbyl palmitate | 0.125 | 0.017% |
| SOLID PHASE | | |
| Fujicalin ® | 450 | 60.37% |
| MCC Avicel PH-102 | 125 | 16.77% |
| Glycine USP | 10 | 1.34% |
| EDTA | 2.5 | 0.34% |
| HPC SSL SF | 10 | 1.34% |
| EtOH | 40 | N/A |
| Ac-Di-Sol | 20 | 2.68% |
| Sodium lauryl sulfate (SLS) | 7.5 | 1.01% |
| Tablet weight | 745.35 | 100.00% |

TABLE 6
Composition of 10 mg CBD SNEDDS TABLET
PER STAGE

| 10 mg tablet | mg/tab |
|---|---|
| OIL PHASE | |
| CBD | 10 |
| Gelucire 44/14 | 5 |
| MCT | 7.2 |
| D-alpha-Tocopheryl acetate | 0.9 |
| D-alpha Tocopherol | 0.36 |
| Soy Lecithin 90G | 0.54 |
| MCT for Lecithin dissolving | 0.54 |
| Kolliphor RH-40 | 4.3 |
| Polysorbate 60 | 8.6 |
| Span 80 | 5.2 |
| Peppermint oil FCC grade | 2.4 |
| Almond oil | 3.0 |
| BHT | 0.05 |
| Ascorbyl palmitate | 0.05 |
| GRANULATION | |
| Fujicalin ® | 115 |
| MCC Avicel PH-102 | |
| Glycine USP | 10 |
| Ac-Di-Sol | 4.0 |
| EDTA | 2.5 |
| 20% HPC SSL-SF in 95% EtOH | 4 |
| EtOH | 16 |
| EXTRAGRANULAR | |
| Fujicalin ® | 115 |
| MCC Avicel PH-102 | 115 |
| Ac-Di-Sol | 15 |
| Glycine USP | 12 |
| Sodium lauryl sulfate (SLS) | 10 |
| Sum | 450.64 |

CUMULATIVE

| 10 mg tablet | mg/tab | % |
|---|---|---|
| OIL PHASE | | |
| CBD | 10 | 2.22% |
| Gelucire 44/14 | 5 | 1.11% |
| MCT | 7.2 | 1.60% |
| D-alpha-Tocopheryl acetate | 0.9 | 0.20% |
| D-alpha Tocopherol | 0.36 | 0.08% |
| Soy Lecithin 90G | 0.54 | 0.12% |
| MCT for Lecithin dissolving | 0.54 | 0.12% |
| Kolliphor RH-40 | 4.3 | 0.95% |
| Polysorbate 60 | 8.6 | 1.91% |
| Span 80 | 5.2 | 1.15% |
| Peppermint oil | 2.4 | 0.53% |
| Almond oil | 3.0 | 0.67% |
| BHT | 0.05 | 0.01% |
| Ascorbyl palmitate | 0.05 | 0.01% |
| SOLID PHASE | | |
| Fujicalin ® | 230 | 51.04% |
| Glycine | 10 | 2.22% |
| Ac-Di-Sol | 19.0 | 4.22% |
| EDTA | 2.5 | 0.55% |
| HPC SSL SF | 4 | 0.89% |
| EtOH | 16 | N/A |
| MCC Avicel PH-102 | 115 | 25.52% |
| Glycine USP | 12 | 2.66% |
| Sodium lauryl sulfate (SLS) | 10 | 2.22% |
| Tablet weight | 450.64 | 100.00% |

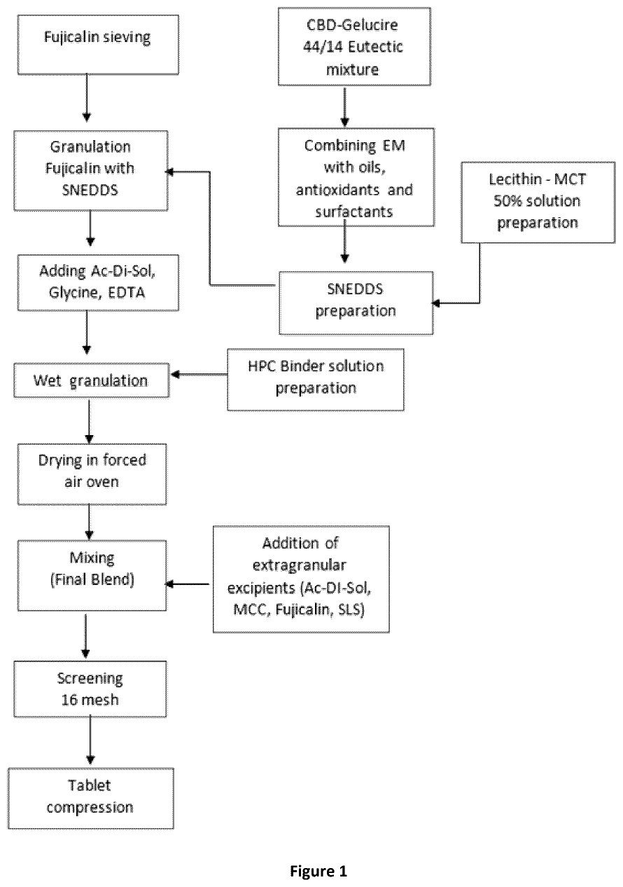

FIG. 1 is a flowchart illustrating the process used for preparing the small-scale batches of compressed tablets for oral administration containing 10 mg, 25 mg, and 50 mg of cannabidiol (CBD). The tablets were prepared by a two-stage wet granulation process, as described in further detail below.

Initially, the oil phase containing the mixture of CBD and Gelucire 44/14 (CBD loaded SNEDDS) was prepared. As noted above, the mixture was prepared by combining Gelucire 44/14 and crystalline CBD, with small amounts of antioxidants, and heating the mixture at 50-55° C. under mixing conditions until the clear liquid was formed. The other oil phase components were then added to the clear liquid.

Then, CBD loaded SNEDDS (oil phase) was adsorbed on Fujicalin® and granulated with a spatula. Prepared SNEDDS was adsorbed on Fujicalin® with intense mixing to ensure complete incorporation of the lipid phase into the pores; the ratio of SNEDDS to Fujicalin was about 1:2.5-1:3.

Next, granulation excipients MCC Avicel PH-102, Glycine USP, Ac-Di-Sol, and EDTA were added to the absorbent-SNEDDS blend, and the mixture was granulated with a 20% solution of low molecular weight hydroxypropyl cellulose (HPC SSL) in ethanol with high shear granulator agitator and chopper with pre-determined mixing times. It was found experimentally that a 20% solution of HPC SSL provides an improved dissolution pattern and result in tablets with higher hardness than if a 10% solution of HPC SSL was used. The HPC SSL forms a film on the oil adsorbed granules, protecting the CBD from oxidation and/or moisture absorption, and also ruptures quickly when it comes in contact with SGF to provide good dissolution patterns.

The formed granulation was dried in the forced air oven for 10-15 minutes at 35° C., mixed with the extragranular excipients, screened through a 16 mesh screen and then tablets were compressed using single punch tablet press at 200 bar (50 mg) and 250 bar (25 and 10 mg tablets) pressure.

FIG. 2 is a photo of exemplary 50 mg, 25 mg, and 10 mg compressed tablets for oral administration (in order from left to right in the photo) prepared according to the processes described herein. The specifications for these tablets are as follows:

TABLE 7
Specifications for laboratory-scale 50
mg, 25 mg, and 10 mg CBD-SNEDDS tablets

| | Total Tablet | Tablet Dimensions | Hardness |
|---|---|---|---|
| CBD Dosage | Weight | and Shape | (kP) |
| 50 mg | ~1150 mg | 18 × 9 mm | ~5 |
| | | capsule shape | |
| 25 mg | ~750 mg | 16 × 9 mm | ~7 |
| | | oval shape | |
| 10 mg | ~450 mg | 16 × 7 mm | ~7 |
| | | capsule shape | |

Dissolution studies were conducted with the 10 mg, 25 mg, and 50 mg tablets using SGF, SIF, and FaSSIF as dissolution media (see Materials & Methods). CBD was completely released from the above-noted formulations in SGF, and up to 60-70% in SIF and FaSSIF. Further, all of the tablets exhibited excellent dissolution behavior in SGF (at least 75%, or at least 80%) CBD released in 15 min at 100 RPM under USP General Chapter <711> conditions—USP Apparatus 2 (Paddle), 37±0.5° C., 900 mL dissolution volume, 5 mL sampling volume, no medium replacement). It should be noted that, prior to quantitation of CBD in the sampled volume, the sample was centrifuged at low speed (e.g. about 1000 RPM) in order to remove any crystalline materials suspended in the solution, to ensure that quantitation of CBD was a true measure of the CBD that is completely dissolved in the discontinuous phase of the formed oil-in-water nanoemulsion. Following centrifugation, 2 mL of supernatant was diluted to 10 mL with methanol prior to quantitation of CBD using a High Performance Liquid Chromatography (HPLC) method that was validated for precision, accuracy, specificity, and linearity. Thus, the tablets exhibited excellent dissolution behaviour, even under these more rigorous sampling conditions.

### Example 3—Preparation of Scale-Up Batches of Compressed Tablets for Oral Administration Containing 10 mg and 25 mg of Cannabidiol (CBD)

Following successful completion of the laboratory-scale batches, scale-up batches of compressed tablets for oral administration containing 10 mg and 25 mg of CBD were then prepared, having a batch size of about 8 kg. Table 8 provides an overview of the composition of the 10 mg and 25 mg tablets of CBD, and specific details having regard to the oil phase (CBD SNEDDS), other intragranular components, and extragranular components are provided in Tables 9 and 10 below.

TABLE 8
Overview of composition of 10 mg and 25 mg CBD tablets

| | | Strength (label claim) | | | |
|---|---|---|---|---|---|
| | | Cannabidiol | | Cannabidiol | |
| | | Tablets, 25 mg | | Tablets, 10 mg | |
| | | Quantity | | Quantity | |
| Component and Quality Standard | | per unit | | per unit | |
| (and Grade, if applicable) | Function | (mg) | % w/w | (mg) | % w/w |
| Cannabidiol (CBD) | Active | 25.00 | 2.38 | 10.00 | 2.22 |
| | Pharmaceutical | | | | |
| | Ingredient (API) | | | | |
| Lauroyl Polyoxyglycerides | Non-ionic | 12.50 | 1.19 | 5.00 | 1.11 |
| (1500), NF (Gelucire ®-44/14) | surfactant HLB | | | | |
| | 11; Eutectic | | | | |
| | mixture component | | | | |
| Butylated Hydroxy Toluene, NF | Antioxidant | 0.13 | 0.01 | 0.05 | 0.01 |
| Ascorbyl Palmitate, NF | Antioxidant | 0.13 | 0.01 | 0.05 | 0.01 |
| Lecithin (Phospholipon ®-90G) | Emulsifier | 1.35 | 0.13 | 0.54 | 0.12 |
| Vitamin E Acetate, USP | Antioxidant | 2.25 | 0.21 | 0.90 | 0.20 |
| (Covitol ® 1360) | | | | | |
| Vitamin E, USP | Antioxidant | 0.90 | 0.09 | 0.36 | 0.08 |
| (Covitol ® F-1490) | | | | | |
| Medium Chain Triglyceride, | Lipid solubilizer | 19.35 | 1.84 | 7.74 | 1.72 |
| NF (Labrafac ™ | | | | | |
| Lipophile WL 1349) | | | | | |
| Polyoxyl 40 Hydrogenated | Hydrophilic non- | 10.75 | 1.02 | 4.30 | 0.96 |
| Castor Oil, NF | ionic surfactant | | | | |
| (Kolliphor ® RH40) | HLB 14.9 | | | | |
| Polysorbate 60, NF | Hydrophilic non- | 21.50 | 2.05 | 8.60 | 1.91 |
| (Kolliphor ® PS60) | ionic surfactant | | | | |
| | HLB 16.0 | | | | |
| Sorbitan Monooleate, NF | Hydrophobic non- | 13.00 | 1.24 | 5.20 | 1.16 |
| [Span ® 80 HP-LQ-(MH)] | ionic surfactant | | | | |
| | HLB 4.3 | | | | |
| Almond Oil, NF | Lipid solubilizer | 7.50 | 0.71 | 3.00 | 0.67 |
| Peppermint Oil, NF | Flavoring agent | 6.00 | 0.57 | 2.40 | 0.53 |
| Anhydrous Dibasic calcium | Absorbent | 696.70 | 66.35 | 230.00 | 51.11 |
| phosphate, USP | | | | | |
| [Fujicalin ® (SG)] | | | | | |
| Microcrystalline Cellulose, | Bulking agent | 172.94 | 16.47 | 114.36 | 25.41 |
| NF (Vivapur ® 102) | (diluent) | | | | |
| Glycine, USP | Bulking agent | 10.00 | 0.95 | 22.00 | 4.89 |
| | (diluent) | | | | |
| Edetate Disodium, USP | Chelating agent | 2.50 | 0.24 | 2.50 | 0.56 |
| (Dissolvine ® Na2-P) | | | | | |
| Hydroxypropyl Cellulose, | Binder | 10.00 | 0.95 | 4.00 | 0.89 |
| USP-NF (HPC-SSL-SFP) | | | | | |
| Croscarmellose Sodium, NF | Super | 20.00 | 1.90 | 19.00 | 4.22 |
| (Vivasol ® GF) | disintegrant | | | | |
| Sodium Lauryl Sulfate, | Surfactant | 7.50 | 0.71 | 10.00 | 2.22 |
| USP-NF (Stepanol WA-100) | | | | | |
| Sodium Stearyl Fumarate, | Lubricant | 10.00 | 0.95 | 0.00 | 0.00 |
| USP-NF | | | | | |

| | | | | |
|---|---|---|---|---|
| Total | 1050.00 | 100.00 | 450.00 | 100.00 |

TABLE 9
Composition of 10 mg CBD tablets
MASTER FORMULA - 10 MG CBD TABLETS
Raw Material Composition
(a) Composition of oil phase of intragranular portion of 10 mg CBD tablets

| | | | |
|---|---|---|---|
| | Formula | Weight | Batch |
| Material Name/Trade Name and | Composition | Composition | Quantity |
| Compendial Reference (if applicable) | (mg/Tablet) | (% w/w)) | (grams) |

Oil Phase Composition

| | | | |
|---|---|---|---|
| Cannabidiol Isolate (CBD)- API | 10.00 | 2.22 | 66.67 |
| Gelucire-44/14 (Lauroyl Polyoxyl-32 | 5.00 | 1.11 | 33.33 |
| Glycerides) EP/NF | | | |
| BHT (Butylated Hydroxy Toluene) | 0.05 | 0.01 | 0.33 |
| USP/NF | | | |
| Ascorbyl Palmitate EP/USP | 0.05 | 0.01 | 0.33 |
| Phospholipon-90G (Soy Lecithin) | 0.54 | 0.12 | 3.60 |
| USP | | | |
| Ethanol (Dehydrated Alcohol USP) | 0.05 | 0.01 | 3.60 |
| Novatol-1360 (D-alpha Tocopherol | 0.90 | 0.20 | 6.00 |
| acetate) USP | | | |
| Novatol-1490 (D-alpha Tocopherol) | 0.36 | 0.08 | 2.40 |
| USP | | | |
| Kollisolv MCT 70 (Medium Chain | 7.20 | 1.60 | 51.60 |
| Triglyceride Oil) EP | | | |
| Kolliphor RH40 (Polyoxyl 40 | 4.30 | 0.96 | 28.67 |
| Hydrogenated Castor oil) USP/EP | | | |
| Kolliphor PS60 (Polysorbate 60) EP | 8.60 | 1.91 | 57.33 |
| Montane 80 PHA Premium (Sorbitan | 5.20 | 1.16 | 34.67 |
| Monooleate) EP | | | |
| Almond Oil | 3.00 | 0.67 | 20.00 |
| Peppermint Oil USP/NF EP | 2.40 | 0.53 | 16.00 |
| Total Oil Phase | 48.14 | 10.70 | 320.93 |

(b) Intragranular and extragranular composition of 10 mg CBD tablets

| | | | |
|---|---|---|---|
| | Formula | Weight | Batch |
| Material Name/Trade Name and | Composition | Composition | Quantity |
| Compendial Reference (if applicable) | (mg/Tablet) | (% w/w) | (grams) |

Intra-granular Composition

| | | | |
|---|---|---|---|
| Fujicalin (Porous Dibasic calcium | 115.00 | 25.56 | 766.67 |
| phosphate) USP | | | |
| Croscarmellose Sodium (Vivasol) | 4.00 | 0.89 | 26.67 |
| USP/NF, EP | | | |
| Glycine USP/EP | 10.00 | 2.22 | 66.67 |
| Dissolvine NA2-P (Ethylenediamine | 2.50 | 0.56 | 16.67 |
| Tetra Acetic Acid Disodium) | | | |
| USP/EP | | | |
| HPC-SSL-SFP (Hydroxy Propyl | 4.00 | 0.89 | 26.67 |
| Cellulose-SSL-SFP low viscosity) | | | |
| Ethanol (Dehydrated Alcohol USP) | 16.00 | 3.56 | 106.67 |
| Intra-granular Total | 135.50 | 30.11 | 903.33 |

Extra-Granular Composition

| | | | |
|---|---|---|---|
| Fujicalin (Porous Dibasic calcium | 115.00 | 25.26 | 766.67 |
| phosphate) USP | | | |
| MCC 102 (Microcrystalline | 114.36 | 25.41 | 762.40 |
| Cellulose 102) USP/NF | | | |
| Croscarmellose Sodium (Vivasol) | 15.00 | 3.33 | 100.00 |
| USP/NF, EP | | | |
| Glycine USP/EP | 12.00 | 2.67 | 80.00 |
| Stepanol-WA100 (Sodium Lauryl | 10.00 | 2.22 | 66.67 |
| Sulfate USP) | | | |
| Extra- Granular Total | 266.36 | 59.19 | 1775.73 |
| TOTAL | 450.00 | 100.00 | 3000.00 |

TABLE 10
Composition of 25 mg CBD tablets
MASTER FORMULA - 25 MG CBD TABLETS
RAW MATERIAL COMPOSITION

| | | | |
|---|---|---|---|
| | Formula | Weight | Batch |
| Material Name/Trade Name and | Composition | Composition | Quantity |
| Compendial Reference (if applicable) | (mg/Tablet) | (% w/w) | (grams) |

(a) Composition of oil phase of intragranular portion of 25 mg CBD tablets
Oil Phase Composition

| | | | |
|---|---|---|---|
| Cannabidiol Isolate (CBD)- API | 25.00 | 2.38 | 71.43 |
| Gelucire-44/14 (Lauroyl | 12.50 | 1.19 | 35.71 |
| Polyoxyl-32 Glycerides) EP/NF | | | |
| BHT (Butylated Hydroxy | 0.13 | 0.01 | 0.37 |
| Toluene) USP/NF | | | |
| Ascorbyl Palmitate EP/USP | 0.13 | 0.01 | 0.37 |
| Phospholipon-90G (Soy | 1.35 | 0.13 | 3.86 |
| Lecithin) USP | | | |
| Ethanol (Dehydrated Alcohol | 1.35 | 0.13 | 3.86 |
| USP) | | | |
| Novatol-1360 (D-alpha | 2.25 | 0.21 | 6.43 |
| Tocopherol acetate) USP | | | |
| Novatol-1490 (D-alpha | 0.90 | 0.09 | 2.57 |
| Tocopherol) USP | | | |
| Kollisolv MCT 70 (Medium | 19.35 | 1.84 | 55.29 |
| Chain Triglyceride Oil) EP | | | |
| Kolliphor RH40 (Polyoxyl 40 | 10.75 | 1.02 | 30.71 |
| Hydrogenated Castor oil) USP/EP | | | |
| Kolliphor PS60 (Polysorbate 60) | 21.50 | 2.05 | 61.43 |
| EP | | | |
| Montane 80 PHA Premium | 13.00 | 1.24 | 37.14 |
| (Sorbitan Monooleate) EP | | | |
| Almond Oil | 7.50 | 0.71 | 21.43 |
| Peppermint Oil USP/NF EP | 6.00 | 0.57 | 17.14 |
| Total Oil Phase | 120.36 | 11.46 | 343.89 |

(b) Intragranular and extragranular composition of 25 mg CBD tablets
Intra-granular Composition

| | | | |
|---|---|---|---|
| Fujicalin (Porous Dibasic calcium | 350.00 | 33.33 | 1000.00 |
| phosphate) USP | | | |
| MCC PH-102 (Microcrystalline | 25.00 | 2.38 | 71.43 |
| Cellulose PH-102) USP/NF | | | |
| Glycine USP/EP | 10.00 | 0.95 | 28.57 |
| Dissolvine Na2-P (Ethylenediamine | 2.50 | 0.24 | 7.14 |
| Tetra Acetic Acid Disodium) USP/EP | | | |
| HPC-SSL-SFP (Hydroxy Propyl | 10.00 | 0.95 | 28.57 |
| Cellulose-SSL-SFP low viscosity) | | | |
| Ethanol (Dehydrated Alcohol USP) | 40.00 | 3.81 | 114.29 |
| Intra-granular Total | 397.50 | 37.86 | 1135.71 |

Extra-Granular Composition

| | | | |
|---|---|---|---|
| Fujicalin (Porous Dibasic calcium | 346.70 | 33.02 | 990.57 |
| phosphate) USP | | | |
| MCC PH-102 (Microcrystalline | 147.94 | 14.09 | 422.69 |
| Cellulose PH-102) USP/NF | | | |
| Croscarmellose Sodium (Vivasol) | 20.00 | 1.90 | 57.14 |
| USP/NF, EP | | | |
| Stepanol-WA100 (Sodium Lauryl | 7.50 | 0.71 | 21.43 |
| Sulfate USP) | | | |
| Sodium Stearyl Fumarate | 10.00 | 0.95 | 28.57 |
| (Lubripharm) | | | |
| Extra- Granular Total | 532.14 | 50.68 | 1520.40 |
| TOTAL | 1050.00 | 100.00 | 3000.00 |

For each of the 10 mg and 25 mg tablet formulations, the required quantity of API (CBD) was calculated based on as is potency from the Certificate of Analysis (COA) if less than 99.5%. Except for Phospholipon 90-G, all semi solid materials were heated to 50° C.±5° C. on a hot plate or oven prior to dispensing. All solid materials were sieved through #30 mesh screen before dispensing. Ethyl Alcohol was evaporated during the process; thus, it was not considered in the total weight of the batch.

### Preparation of Oil Phase Composition:

Gelucire 44/14 was heated to a temperature of about 50-65° C. under stirring conditions. Once the Gelucire 44/14 had melted, the temperature of the product was maintained at about 50° C. and butylated hydroxy toluene and ascorbyl palmitate were added thereto. The resulting composition was mixed for about 5 minutes or until blended. CBD was added to the composition while maintaining the temperature of the composition at about 50° C., and mixing was continued for about 5-8 minutes to at least partially dissolve the CBD. The mixing vessel was covered to protect the CBD from light and oxidation. Peppermint oil, Kollisolv MCT 70, Kolliphor RH 40, Kolliphor PS 60, Montane 80 PHA, almond oil, Novatol-1360, and Novatol-1490 were then added under mixing conditions, while heating was continued to maintain the temperature of the mixture at about 40-45° C. Mixing was continued for 10-20 minutes until all components were completely dissolved. The mixture was then cooled slightly to about 40° C. A pre-mixed solution of Phospholipon-90G in ethanol was then added under mixing conditions, and mixing was continued for about 8-12 minutes while heating to maintain the solution at about 40° C.

### Preparation of Intragranular Composition

The polymeric binder solution was prepared by gradually adding Hydroxy Propyl Cellulose-SSL-SFP to the ethanol under mixing conditions. Mixing was continued for 60-90 minutes until the Hydroxy Propyl Cellulose-SSL-SFP was completely dissolved.

Fujicalin was coated with the oil phase using a Glatt-Tabletop Mixer Granulator (TMG) granulator (or similar apparatus), with an agitator speed of 200-500 RPM and a chopper speed of 780-900 RPM. Fujicalin was mixed alone for about 1 minute, prior to adding the oil phase over a period of about 2-3 minutes, with continued mixing for a few minutes thereafter. MCC PH-102 (25 mg tablet)/Vivasol (10 mg tablet), Dissolvine NA2, and glycine were then added, and mixing was continued for about 2 minutes.

The polymeric binding solution was then added to the granulator, with the agitator speed and/or chopper speed raised as appropriate. Mixing was continued for a few minutes until the wet granules were smooth and free flowing.

The wet granules were dried using a Midi Glatt Fluidized Bed Dryer (FBD) at a temperature of about 20° C. The dried granules were then sifted through a #20 sieve.

### Incorporation of Extragranular Materials and Tablet Preparation

Components of the extragranular composition were added to the above-noted dried granules under blending conditions (e.g. at a speed of about 25 RPM). Tablets were then prepared using a tablet press (e.g. IMA PREXIMA-300, or similar). The 10 mg CBD tablet was prepared with 10.00 mm Round standard concave upper punch, lower punches and dies, whereas the 25 mg CBD tablet was 8 mm×10 mm capsule-shaped.

The 10 mg CBD tablets had a target tablet hardness of about 6.0 kP (5.0 kP-9.0 kP range), a target tablet thickness of about 5.00 mm (range of 4.50 mm-5.50 mm), and a targeted friability loss of not more than 1%, with the desired appearance being plain white to off white tablets with minor mottling.

The 25 mg CBD tablets had a target tablet hardness of about 10.0 kP (6.0 kP-18.0 kP range), a target tablet thickness of about 5.35 mm (range of 4.85 mm-5.85 mm), and a targeted friability loss of not more than 1%, with the desired appearance being plain white to off white tablets with minor mottling.

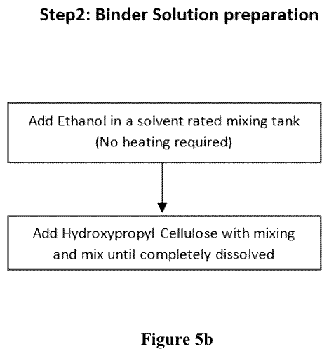

FIG. 4 is a photo of exemplary 25 mg and 10 mg compressed tablets for oral administration (in order from left to right in the photo) prepared according to the present example. The above-noted tablet targets were met and dissolution studies were then conducted.

### Dissolution Testing

Dissolution studies were conducted with the 10 mg and 25 mg tablets using SGF as dissolution media (see Materials & Methods). Dissolution studies were conducted according to USP General Chapter <711> testing procedures, using the following parameters:

TABLE 11
Dissolution studies parameters for scale-up
10 mg and 25 mg CBD tablets

| Apparatus | USP Apparatus 2 (Paddle) | |
|---|---|---|
| Speed | 100 | rpm |
| Temperature | 37 ± 0.5° | C. |
| Dissolution medium | Simulated Gastric Fluid without Enzyme | |
| Dissolution volume | 900 | mL |
| Sampling time points | 15, 30, 45, and 60 minutes | |
| Sampling Volume | 5 | mL |

| | |
|---|---|
| Medium replacement | No |
| Sample Treatment | Centrifuge the sample for 10 minutes at |
| | 1000 rpm, take 2 mL of supernatant |
| | and dilute to 10 mL with Methanol |

It should be noted that a centrifugation step was incorporated into the dissolution studies outlined in the present example, similar to Example 2, again to ensure that quantitation of CBD was a true measure of the CBD that is completely dissolved in the discontinuous phase of the formed oil-in-water nanoemulsion. CBD was quantitated using the HPLC method as outlined in Example 2.

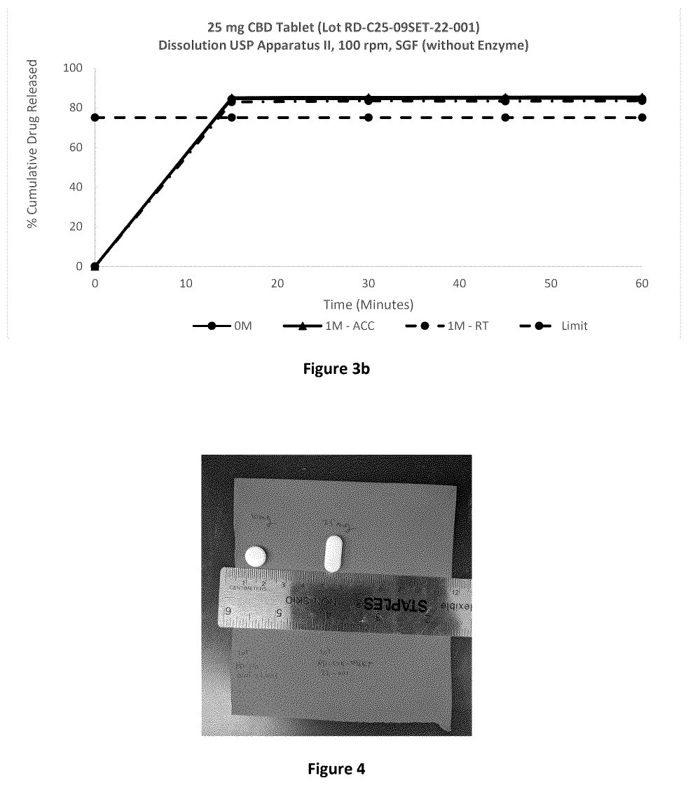

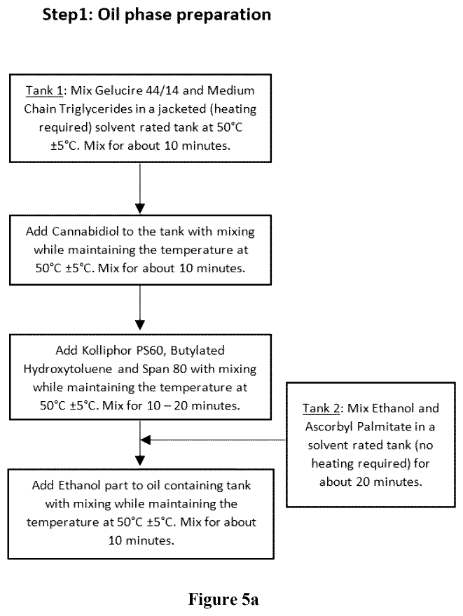

As shown in FIGS. 3a and 3b, the tablets exhibited excellent dissolution behaviour, even under these more rigorous sampling conditions.

FIG. 3a is a chart illustrating dissolution behaviour of 10 mg CBD tablets following 2 months of storage under room temperature (“RT”; 25° C./60% RH) or accelerated storage conditions (“Acc”; 40° C. 75% RH). In each instance, at least 75%, and in fact more than 80%, of the CBD was released (i.e. dissolved) within 15 minutes. The order of the plot lines at the 60-minute mark is as shown in the graph legend, with the “2M-RT” sample exhibiting slightly higher dissolution of CBD than the “2M-Acc” sample.

FIG. 3b is a chart illustrating dissolution behaviour of 25 mg CBD tablets following 0 months and 2 months of storage under room temperature (“RT”; 25° C./60% RH) or accelerated storage conditions (“Acc”; 40° C. 75% RH). In each instance, at least 75%, and in fact more than 80%, of the CBD was released (i.e. dissolved) within 15 minutes. The order of the plot lines at the 60-minute mark is as shown in the graph legend, with the differences in dissolution between these samples being very slight.

### Nanoemulsion Studies

Emulsions released from CBD tablets (10 & 25 mg) during dissolution in SGF were investigated. Each tablet was placed into a separate glass vessel, containing SGF with or without 0.1% Polysorbate-80 (Tween-80) at 37° C. and dissolved for 30 minutes. Samples of the dissolution media were then taken and particle size was measured using Malvern Zetasizer™ ZS. Samples were filtered through a 0.45 mcm PTFE (Polytetrafluoroethylene) membrane syringe filter before size determination, to remove larger particles which corresponded to tablet excipients (e.g. cellulose derivatives). To assure stability of the formed emulsion in presence of SGF a small amount of Polysorbate-80 (0.1% Tween-80) was added to the dissolution media. Results are shown in Table 12 below.

TABLE 12
Nanoemulsion size determination for scale-up 10 mg and 25 mg CBD tablets

| | | | | Peak size - |
|---|---|---|---|---|
| | | Peak size - | Z-average | volume for |
| | Z-average | volume for | 37° C. SGF + | 37° C. SGF + |
| | 37° C. SGF | 37° C. SGF | 0.1% Tween-80 | 0.1% Tween-80 |
| RD-C10-01OT-21-003 | 172 nm | 195 ± 73 nm | 138 nm | 155 ± 42 nm |
| (10 mg tablet) | PdI 0.176 | | PdI 0.193 | |
| RD-C25-01SET-22-001A | 143 nm | 174 ± 55 nm (64%) | 131 nm | 144 ± 40 nm |
| (25 mg tablet) | PdI 0.132 | 89 ± 13 nm (36%) | PdI 0.166 | |

Thus, the scale-up 10 mg and 25 mg CBD tablets release nanoemulsions upon dissolution in SGF.

### Example 4—Alternative Process for Preparation of Lab-Scale and Scale-Up Batches of Compressed Tablets for Oral Administration Containing 10 mg and 25 mg of Cannabidiol (CBD)

The tablets of Example 3 exhibited excellent dissolution behaviour and acceptable durability and bioavailability. However, this embodiment of the composition contained a large number of excipients, including multiple excipients in the same functional category. A smaller tablet size with fewer excipients was deemed desirable for user compliance and from a cost perspective. In addition, it was considered that removal of some of the excipients that may be allergenic to potential patients (e.g. lecithin and almond oil) would be beneficial. Finally, it was considered that adjustments to the composition to reduce the amount of oil present could reduce the amount of solid carrier required, thereby further reducing tablet size and improving on tablet durability with further scale-up. The present example outlines an alternate embodiment of the pharmaceutical composition wherein the composition has been further optimized to meet these goals.

Table 13 provides an overview of the composition of the 10 mg and 25 mg tablets of CBD prepared according to the process of the present example. A lab-scale batch of approximately 3 kg was prepared, followed by a scale-up batch of approximately 6.5 kg using the same equipment setup. Overall, the critical quality attributes (e.g. hardness, dissolution properties, friability, average weight, etc.) of the drug product were comparable. The tablet properties presented below (hardness, dissolution properties, and nanoemulsion/particle size data) relate to the 3 kg batch.

TABLE 13
Overview of composition of 10 mg and 25 mg CBD tablets prepared
according to alternative process of present example

| | Amount | |
|---|---|---|
| | (mg/tab) | |
| Ingredients | 10 mg | 25 mg |
| Oil Phase | | |
| Cannabidiol - API | 10.00 | 25.00 |
| Lauroyl Polyoxyl-32 Glycerides (Gelucire 44/14) - | 0.625 | 1.563 |
| solubilizing agent | | |
| Butylated Hydroxytoluene - antioxidant | 0.640 | 1.600 |
| Ascorbyl Palmitate - antioxidant | 0.640 | 1.600 |
| Ethyl Alcohol* | 6.000 | 15.00 |
| Medium Chain Triglycerides (Labrafac Lipophile | 0.968 | 2.420 |
| WL 1349) - carrier oil | | |
| Polysorbate 60 (Kolliphor PS 60) - emulsifying agent | 12.90 | 32.25 |
| Sorbitan monooleate (Span 80) - emulsifying agent | 5.200 | 13.00 |
| Binder Solution (20% w/w Solid Content) | | |
| Hydroxypropyl cellulose low viscosity | 17.50 | 43.75 |
| (HPC SSL-SFP) - polymeric binder | | |
| Ethyl Alcohol* | 70.00 | 175.0 |
| Intra-granular | | |
| Porous Dibasic Calcium Phosphate (Fujicalin SG) - | 136.4 | 341.1 |
| porous solid carrier | | |
| Microcrystalline Cellulose 102 (Vivapur 102) - | 60.24 | 150.6 |
| intragranular diluent | | |
| Hydroxypropyl cellulose low viscosity | 10.50 | 26.25 |
| (HPC SSL-SFP) - polymeric binder | | |
| Croscarmellose sodium (Vivasol GF) - disintegrant | 7.000 | 17.50 |
| Extra-granular | | |
| Microcrystalline Cellulose 102 (Vivapur 102) - | 45.36 | 113.4 |
| extragranular diluent | | |
| Croscarmellose sodium (Vivasol GF) - disintegrant | 21.00 | 52.50 |
| Sodium Stearyl Fumarate (Lubripharm SSF) - lubricant | 21.00 | 52.50 |
| Total (Tablets): | 350.0 | 875.0 |

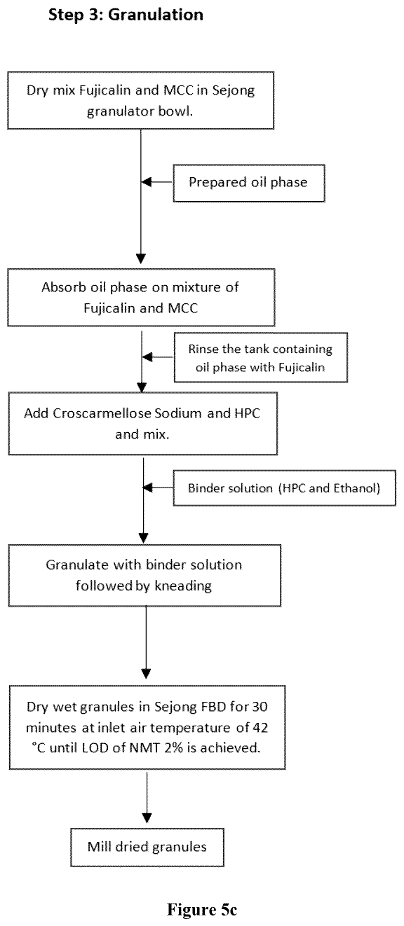

FIGS. 5a to 5d set out the detailed process parameters for preparation of the pharmaceutical composition of the present example. In these figures, the acronym “LOD” represents “loss on drying”, the acronym “NMT” refers to “not more than”, and the acronym “FBD” refers to “fluidized bed dryer”.

TABLE 14
Specifications for 25 mg, and 10 mg CBD-SNEDDS tablets

| | Total Tablet | Tablet Dimensions | Hardness |
|---|---|---|---|
| CBD Dosage | Weight | and Shape | (kP) |
| 25 mg | ~875 mg | 18 × 8 mm | ~9 |
| | | capsule shape | |
| 10 mg | ~350 mg | 13 × 6 mm | ~6 |
| | | modified oval shape | |

### Dissolution Testing

Dissolution studies were conducted as described in Example 3, except that sampling time points were 15 and 30 minutes, and there was no extended storage of the tablets prior to testing. Specifically, dissolution studies were conducted with the 10 mg and 25 mg tablets using SGF as dissolution media (see Materials & Methods), according to USP General Chapter <711> testing procedures, using the following parameters:

TABLE 15
Dissolution studies parameters for 10 mg and 25 mg CBD tablets

| Apparatus | USP Apparatus 2 (Paddle) | |
|---|---|---|
| Speed | 100 | rpm |
| Temperature | 37 ± 0.5° | C. |
| Dissolution medium | Simulated Gastric Fluid without Enzyme | |
| Dissolution volume | 900 | mL |
| Sampling time points | 15 and 30 minutes | |
| Sampling Volume | 5 | mL |

| | |
|---|---|
| Medium replacement | No |
| Sample Treatment | Centrifuge the sample for 10 minutes at |
| | 1000 rpm, take 2 mL of supernatant |
| | and dilute to 10 mL with Methanol |

As with Examples 2 and 3, a centrifugation step was incorporated into the dissolution studies to ensure that quantitation of CBD via the above-noted HPLC method was a true measure of the CBD that is completely dissolved in the discontinuous phase of the formed oil-in-water nanoemulsion.

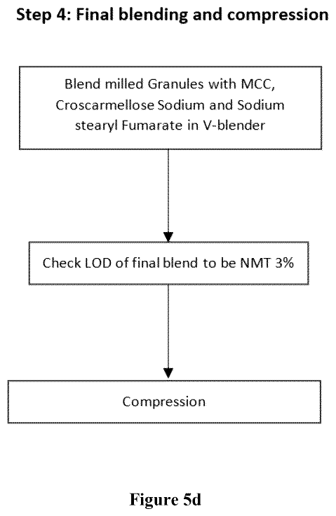

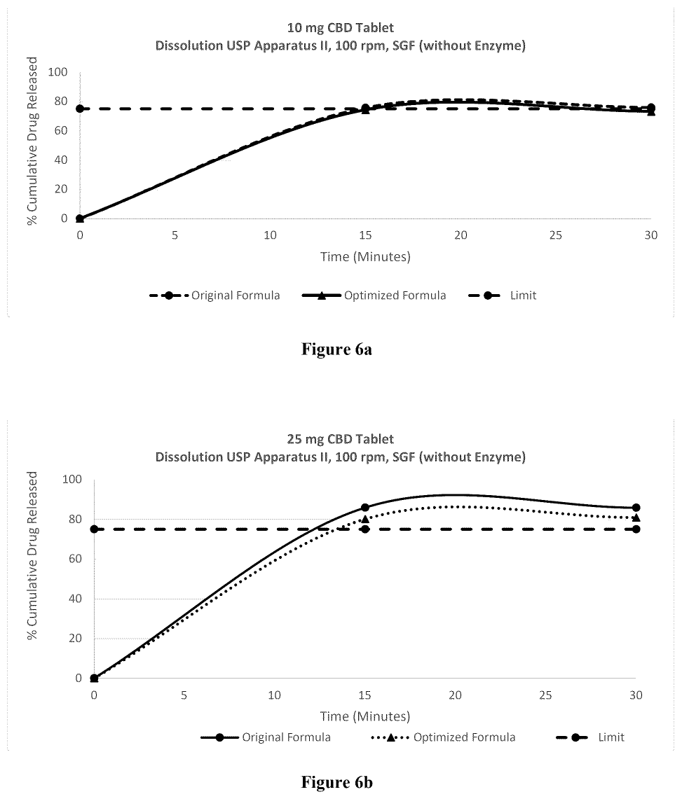

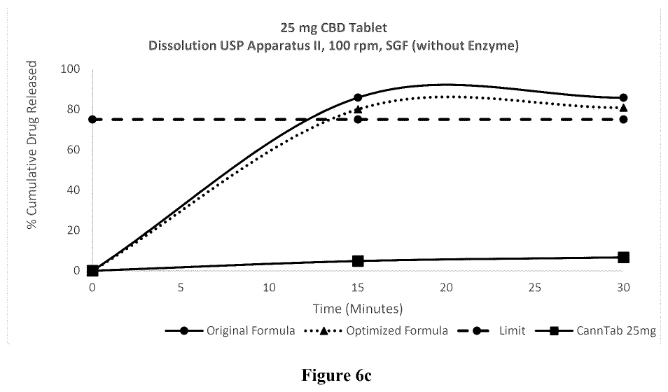

FIGS. 6a and 6b compare the dissolution behaviour of 10 mg CBD tablets and 25 mg CBD tablets prepared according to Example 3 (“Original Formula”) and Example 4 (“Optimized Formula”). Based on this data, it can be concluded that the original and the optimized formulations exhibit comparable dissolution profiles.

FIG. 6c compares the dissolution behaviour of 25 mg CBD tablets prepared according to Example 3 (“Original Formula”) and Example 4 (“Optimized Formula”) with INSTACANN® 25 mg CBD tablets manufactured by CannTab Therapeutics Limited, under the above-noted conditions. According to the label, the INSTACANN® 25 mg CBD tablets contained microcrystalline cellulose, precipitated silica, povidone K-30, Betadex, modified food starch, Labrasol©, crospovidone, magnesium stearate, ascorbyl palmitate, D-α-tocopherol polyethylene glycol 1000 succinate, medium chain triglyceride, hypromellose, polyethylene glycol, talc, titanium dioxide & Quinoline Yellow Lake. FIG. 6c illustrates the superior dissolution characteristics of the 25 mg CBD tablets of Examples 3 and 4 versus the INSTACANN® 25 mg CBD tablets in SGF (no enzyme), which is considered as an in vivo performance relevant medium for immediate release drug products.

### Nanoemulsion Studies

Each tablet was placed into a separate vessel, containing SGF at 37° C. and dissolved for 30 minutes followed by centrifugation. Samples were filtered through a 0.45 mcm PTFE membrane syringe filter before size determination, to remove larger particles which corresponded to tablet excipients (e.g. cellulose derivatives). Particle size was measured using Malvern Zetasizer™ ZS.

Results are shown in Table 16 below.

TABLE 16
Particle size determination (nanometer) for original
(Example 3) and optimized (Example 4) formulations

| | Cannabidiol | Cannabidiol | Cannabidiol | Cannabidiol |
|---|---|---|---|---|
| | Tablets 25 mg | Tablets 25 mg | Tablets 10 mg | Tablets 10 mg |
| | (Example 3) | (Example 4) | (Example 3) | (Example 4) |
| Z-average (d · nm) | 177.6 | 200.9 | 175.6 | 217.9 |
| Polydispersity | 0.207 | 0.190 | 0.161 | 0.225 |
| index (PDI) | | | | |

The pharmaceutical composition according to Example 4 therefore successfully removed a number of excipients, including allergens and redundant excipients, relative to the composition of Example 3, while maintaining excellent dissolution properties and improving upon tablet durability. The composition of Example 4 also results in a smaller tablet for better patient compliance and cost competitiveness.

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. The scope of the claims should not be limited to the preferred embodiments set for the description, but should be given the broadest interpretation consistent with the description as a whole.

1-30. (canceled)

31. A pharmaceutical composition in the form of a compressed tablet for oral administration having an intragranular portion and an extragranular portion, wherein:
the intragranular portion comprises:
a first porous solid carrier comprising a physiologically acceptable salt that is soluble in gastric fluid or in an acidic aqueous media;
a self-nanoemulsifying drug delivery system (SNEDDS) adsorbed on the carrier, wherein the SNEDDS comprises:
at least one cannabinoid;
one or more solubilizing agents selected from physiologically acceptable organic compounds comprising: alcohols C8-C18, cyclic alcohols, aromatic alcohols, glycerides, polyethylene glycols, polyethylene glycol esters, aromatic esters, phenols, tocopherols, phospholipids, polyoxylglycerides, or polyoxyethylene stearates;
one or more emulsifying agents selected from physiologically acceptable nonionic surfactants; and
one or more carrier oils; and
the extragranular portion comprises:
an optional second porous solid carrier comprising a physiologically acceptable salt that is soluble in gastric fluid or in an acidic aqueous media;
one or more disintegrants;
one or more diluents; and
one or more lubricants.

32. The pharmaceutical composition of claim 31, wherein the first porous solid carrier and/or second porous solid carrier comprises porous dibasic calcium phosphate or porous tribasic calcium phosphate.

33. The pharmaceutical composition of claim 31, wherein the first porous solid carrier and the second porous solid carrier comprise porous dibasic calcium phosphate, wherein the dibasic calcium phosphate is anhydrous.

34. The pharmaceutical composition of claim 31, wherein the one or more solubilizing agents are selected from glycerides, lauroyl polyoxylglycerides or caprylocaproyl polyoxylglycerides.

35. The pharmaceutical composition of claim 31, wherein the one or more solubilizing agents are selected from glyceryl monolinoleate, lauroyl polyoxyl-32 glycerides, caprylocaproyl polyoxyl-8 glycerides, or polyoxyl-32 stearate.

36. The pharmaceutical composition of claim 31, wherein the one or more solubilizing agents is lauroyl polyoxyl-32 glycerides.

37. The pharmaceutical composition of claim 31, wherein the one or more emulsifying agents are selected from lecithin, polyoxyl 40 hydrogenated castor oil, polysorbate 60, sorbitan monooleate, polysorbate 20, polysorbate 80, or sorbitan monostearate.

38. The pharmaceutical composition of claim 31, wherein the one or more carrier oils are selected from medium-chain triglycerides (MCT), almond oil, peppermint oil, fractionated coconut oil, sesame oil, pumpkin seed oil, avocado oil, olive oil, corn oil, soybean oil, cottonseed oil, safflower oil, L-menthol-containing oil, or spearmint oil.

39. The pharmaceutical composition of claim 31, wherein the intragranular portion comprises one or more antioxidants.

40. The pharmaceutical composition of claim 39, wherein the one or more antioxidants are selected from butylated hydroxytoluene, butylated hydroxyanisole, ascorbic acid, ascorbyl palmitate, tocopherols and tocopherol esters, propyl gallate, or tertiary butyl hydroquinone.

41. The pharmaceutical composition of claim 31, wherein the intragranular portion further comprises one or more of an intragranular diluent, and a chelating agent.

42. The pharmaceutical composition of claim 41, wherein the intragranular portion comprises an intragranular diluent selected from microcrystalline cellulose (MCC), polyols, polysaccharides, starches, sugars, or amino acids, or combinations thereof.

43. The pharmaceutical composition of claim 42, wherein the intragranular diluent is selected from glycine, microcrystalline cellulose, or combinations thereof.

44. The pharmaceutical composition of claim 41, wherein the intragranular portion comprises a chelating agent selected from ethylenediaminetetraacetic acid (EDTA) and EDTA salts, ethylene glycol-bis(O-aminoethyl ether)-N,N,N′,N′-tetraacetic acid (EGTA) and EGTA salts, citric acid and citrates, or combinations thereof.

45. The pharmaceutical composition of claim 31, wherein the intragranular portion comprises a polymeric binder, applied on an intragranular admixture comprising the first porous solid carrier having the SNEDDS adsorbed thereto; the binder comprising a film-forming agent selected from low viscosity hydroxylpropyl cellulose, or hydroxyethyl cellulose;
optionally, wherein the intragranular admixture comprises a further polymeric binder in admixture with the first porous solid carrier, wherein the further polymeric binder is selected from selected from low viscosity hydroxylpropyl cellulose, or hydroxyethyl cellulose.

46. The pharmaceutical composition of claim 31, wherein the intragranular portion optionally comprises one or more intragranular disintegrants, and wherein the one or more intragranular disintegrants and the one or more disintegrants in the extragranular portion are independently selected from croscarmellose sodium or sodium starch glycolate.

47. The pharmaceutical composition of claim 31, wherein the one or more diluents in the extragranular portion are selected from microcrystalline cellulose (MCC), polyols, polysaccharides, starches, sugars, or amino acids.

48. The pharmaceutical composition of claim 31, wherein the one or more lubricants are selected from sodium lauryl sulfate, polyethylene glycol (PEG) 3350, PEG 6000, PEG 8000, or sodium stearyl fumarate.

49. The pharmaceutical composition of claim 31, wherein the pharmaceutical composition is essentially free from silicon dioxides, silicates, polyvinylpyrrolidone (PVP) polymers, or copolymers of polyvinylpyrrolidone.

50. The pharmaceutical composition of claim 31, wherein:
the at least one cannabinoid is present in an amount of from about 1% w/w to about 10% w/w of the tablet, or of from about 2% w/w to about 4% w/w of the tablet;
the first porous solid carrier and the second porous solid carrier, if present, are collectively present in an amount of from about 30% w/w to about 75% w/w of the tablet, or of from about 35% w/w to about 75% w/w of the tablet, or of from about 40% w/w to about 75% w/w of the tablet, or of from about 35% w/w to about 45% w/w of the tablet;
the one or more solubilizing agents are collectively present in an amount of from about 0.1% w/w to about 5% w/w of the tablet, or of from about 0.5% w/w to about 5% w/w of the tablet, or of from about 0.1% w/w to about 0.3% w/w of the tablet;
the one or more emulsifying agents are collectively present in an amount of from about 2% w/w to about 15% w/w of the tablet, or of from about 4% w/w to about 6% w/w of the tablet;
the one or more carrier oils are collectively present in an amount of from about 0.1% w/w to about 10% w/w of the tablet, or of from about 2% w/w to about 10% w/w of the tablet, or of from about 0.2% w/w to about 0.4% w/w of the tablet;
the intragranular portion comprises one or more intragranular disintegrants, and the one or more intragranular disintegrants and the one or more disintegrants in the extragranular portion are collectively present in an amount of from about 1% w/w to about 10% w/w of the tablet, or of from about 7% w/w to about 9% w/w of the tablet;
the intragranular portion comprises one or more intragranular diluents, and the one or more intragranular diluents and the one or more diluents in the extragranular portion are collectively present in an amount of from about 5% w/w to about 40% w/w of the tablet, or of from about 25% w/w to about 35% w/w of the tablet; and
the one or more lubricants are collectively present in an amount of from about 1% w/w to about 10% w/w of the tablet, or of from about 1% w/w to about 5% w/w of the tablet, or of from about 1% w/w to about 7% w/w of the tablet, or of from about 5% w/w to about 7% w/w of the tablet.

51. The pharmaceutical composition of claim 50, wherein the pharmaceutical composition further comprises the polymeric binder and, optionally, the further polymeric binder as defined in claim 15, wherein the polymeric binder and the further polymeric binder are collectively present in an amount of from about 0.5% w/w to about 10% w/w of the tablet, or of from about 5% w/w to about 10% w/w of the tablet; or of from about 7% w/w to about 9% w/w of the tablet.

52. The pharmaceutical composition of claim 31, wherein a dissolution rate of the at least one cannabinoid in the compressed tablet for oral administration is such that, when tested using USP Apparatus II (paddles) set to a rotation speed of 100 rpm in 900 mL of simulated gastric fluid with no enzyme at 370° C., at least 70% of the at least one cannabinoid dissolves in 30 minutes or less, or at least 75% of the at least one cannabinoid dissolves in 30 minutes or less.

53. The pharmaceutical composition of claim 52, wherein at least 70% of the at least one cannabinoid dissolves in 15 minutes or less, or at least 75% of the at least one cannabinoid dissolves in 15 minutes or less.

54. The pharmaceutical composition of claim 31, wherein the at least one cannabinoid is present in the compressed tablet an amount of from about 5 mg to about 100 mg.

55. A process for preparing the pharmaceutical composition of claim 31, the process comprising:
combining the at least one cannabinoid and the one or more solubilizing agents under heating conditions to at least partially solubilize the at least one cannabinoid, forming a first mixture;
combining the one or more emulsifying agents, and one or more carrier oils with the first mixture under heating conditions, to form the SNEDDS;
combining the SNEDDS with the first porous solid carrier to adsorb the SNEDDS to the first porous solid carrier;
combining the first porous solid carrier having the SNEDDS adsorbed thereon with the second porous solid carrier, if present, the one or more disintegrants, the one or more diluents, and the one or more lubricants, to form a pre-tabletting mixture; and
forming the compressed tablet from the pre-tabletting mixture.

56. A process for preparing the pharmaceutical composition of claim 31, the process comprising:
combining the at least one cannabinoid, the one or more solubilizing agents, and the one or more carrier oils under heating conditions to at least partially solubilize the at least one cannabinoid, forming a first mixture;
combining the one or more emulsifying agents with the first mixture under heating conditions, to form the SNEDDS;
combining the SNEDDS with the first porous solid carrier to adsorb the SNEDDS to the first porous solid carrier;
combining the first porous solid carrier having the SNEDDS adsorbed thereon with the second porous solid carrier, if present, the one or more disintegrants, the one or more diluents, and the one or more lubricants, to form a pre-tabletting mixture; and
forming the compressed tablet from the pre-tabletting mixture.

57. The process of claim 56, wherein the second porous solid carrier is absent.

58. The pharmaceutical composition of claim 31, wherein the second porous solid carrier is absent.

59. The pharmaceutical composition of claim 31, wherein the at least one cannabinoid is selected from cannabidiol (CBD), delta-9-tetrahydrocannabinol (Δ9-THC), cannabigerol (CBG), or mixtures thereof.

60. The pharmaceutical composition claim 31, wherein the at least one cannabinoid is CBD.
